SEQUENCE LISTING (18 sequences)

SEQ ID NO: 1 - DNA, 1720 bp, Mycobacterium tuberculosis
tcagtgcgcgtgcccgtggtgatggtcgtgatcttctgccttggccggcttgtcgaccacgaccgtctcggtggtgagtaccatccgggcaaccgatgacgcgttcaacaccgccgacctagtcaccttgaccgggtcgatgacgccgtcagcggccaagtcaccatagctcagggtgttcacgttcagcccatgcccggcgggtagctcgctgaccttgttgaccaccaccgagccgtccaagccagcgttggcggcgatccagaacaacggcgcggcaagggcttcggagaacacgtcgacaccgaggacctcgtcaccggtcagcgacgcacgcagttcggtcagcgccttgcgggcctggtggatgagcgaggctcccccaccagggacgatgccctcctcgaccgcggccttggcggccgcgaccgcatcctcgacgctttccttgcgctccttgagtgcggtctcggtggcggcacccaccttgatgacagcaaccccgccggccagtttggccagccgctcgccaagcttttcccgatcccaatccgaatcgctcttgtcgatctcggcacgcaagtgcttcgcccggttggccaccgcttctgcggtgccgccgccgtcgacaatgaccgtgtcgtccttgctgaccaccacgcgtcgggccgagcccagcacctccaagcccacctcgcgcagcaccatgccggcgtcggggttgaccacctggccacccgtcaccaccgccaggtcctcaaggaacgccttacggcggtcaccgaagtacggccccttgaccgcgaccgctttcaacgtcttgcgaatcgcgttgacgaccagcgtcgccaacgcttcgccctccacgtcttcagccacgatcagtagtggcttacccgttcctgcaaccttttccagcaatggcaacagatcgggaagcgagctgatcttgtcttggtgcagcaggatcaacgcgtcctcgagcaccgcctgctggttatcgaagtcggtaacgaagtatgccgacaagaagcccttgtcgaagccgataccctcggtgaactccaactcggtgcccagcgtcgaggattcttcgacgctgaccacgccgtcgtggccgaccttgctcatcgcttcgccaaccaggtcaccgatctgctcgtcgcgcgaggacaccgtcgccacctgcgcgatgccggtcttgccggacaccggcgtggccgatgccagcagcgcctcggataccgcgtcggcggccttgccgattcccacgccgagcgcgatcgggttgacgccggcggccactagcctcaggccgcccttgatcagtgcctgcgccaagatggttgcggtggtggtgccgtcaccggccacatcgttggtcttggtggccaccgacttcaccagctgggcgcccaagtcttcaaacggatcttccagctcgatctcacgtgccaccgtgacgccgtcgttggtaaccgtgggtccgccaaacgccttggccagcaccacatgccggccgcgcggccccagcgtcacccgcacggtgtcggccagcttgtccatgccgacctccatggcgcgacgcgcggtttcgtcgtattcgatcagcttgctcatcaggctcctctacgcagggctagtccgctaacgcatgccgccccggaaatcacccgtggtgagcacggggatcgccggggcggaacacgctctactactt

SEQ ID NO: 2 - DNA, 333 bp, Artificial Sequence
  Target sequence
tcagtgcgcgtgcccgtggtgatggtcgtgatcttctgccttggccggcttgtcgaccacgaccgtctcggtggtgagtaccatccgggcaaccgatgacgcgttcaacaccgccgacctagtcaccttgaccgggtcgatgacgccgtcagcggccaagtcaccatagctcagggtgttcacgttcagcccatgcccggcgggtagctcgctgaccttgttgaccaccaccgagccgtccaagccagcgttggcggcgatccagaacaacggcgcggcaagggcttcggagaacacgtcgacaccgaggacctcgtcaccggtcagcgacgc

SEQ ID NO: 3 - DNA, 21 bp, Artificial Sequence
  Target sequence
tcaacaccgccgacctagtca

SEQ ID NO: 4 - DNA, 20 bp, Artificial Sequence
  Target sequence
cttcggagaacacgtcgaca

SEQ ID NO: 5 - DNA, 24 bp, Artificial Sequence
  Target sequence
tcagggtgttcacgttcagcccat

SEQ ID NO: 6 - DNA, 28 bp, Artificial Sequence
  Target sequence
gacgcgttcaacaccgccgacctagtca

SEQ ID NO: 7 - DNA, 21 bp, Artificial Sequence
  Target sequence
cttcggagaacacgtcgacac

SEQ ID NO: 8 - DNA, 22 bp, Artificial Sequence
  Target sequence
aacacgtcgacaccgaggacct

SEQ ID NO: 9 - DNA, 21 bp, Artificial Sequence
  Oligonucleotide probe
agttgtggcggctggatcagt

SEQ ID NO: 10 - DNA, 20 bp, Artificial Sequence
  Oligonucleotide probe
gaagcctcttgtgcagctgt

SEQ ID NO: 11 - DNA, 24 bp, Artificial Sequence
  Oligonucleotide probe
agtcccacaagtgcaagtcgggta

SEQ ID NO: 12 - DNA, 28 bp, Artificial Sequence
  Oligonucleotide probe
ctgcgcaagttgtggcggctggatcagt

SEQ ID NO: 13 - DNA, 21 bp, Artificial Sequence
  Oligonucleotide probe
gaagcctcttgtgcagctgtg

SEQ ID NO: 14 - DNA, 22 bp, Artificial Sequence
  Oligonucleotide probe
ttgtgcagctgtggctcctgga

SEQ ID NO: 15 - DNA, 20 bp, Artificial Sequence
  Left-end primer
cttctgccttggccggcttg

SEQ ID NO: 16 - DNA, 20 bp, Artificial Sequence
  Right-end primer
gcaaggcgctgaccgaactg

SEQ ID NO: 17 - DNA, 20 bp, Artificial Sequence
  Left-end primer
ttgaccaccaccgagccgtc

SEQ ID NO: 18 - DNA, 20 bp, Artificial Sequence
  Right-end primer
tggacaagctggccgacacc

(19) United States
(12) Patent Application Publication
(10) Pub. No.: US 2006/0073475 A1
(43) Pub. Date: Apr. 6, 2006

(54) Compositions and methods for detecting pathogenic bacteria expressing chaperonin proteins

(76) Inventors: Nanibhushan Dattagupta, San Diego, CA (US); Ketan Shah, San Diego, CA (US)

Correspondence Address: BUCHANAN INGERSOLL LLP;(INCLUDING BURNS, DOANE, SWECKER & MATHIS), 12230 EL CAMINO REAL, SUITE 300, SAN DIEGO, CA, 92130, US

(21) Appl. No.: 10/216,488

(22) Filed: Aug. 9, 2002

(51) Int. Cl. C12Q 1/68; C07H 21/04

(52) U.S. Cl. 435/6; 536/24.1

(57) ABSTRACT

This invention relates generally to compositions and methods for detection of pathogenic bacteria that express extracellular chaperonin proteins. In one embodiment, it relates to Mycobacterium tuberculosis (Mtb) detection and provides for novel probes for a specific and sensitive diagnostic test for Mycobacterium tuberculosis complex (TBC) that hybridize with the groEL-1 gene. Arrays comprising the novel probes immobilized on a support for hybridization analysis and methods for TBC detection using the probes are also provided.

### FIELD OF THE INVENTION

This invention relates generally to compositions and methods for detection of pathogenic bacteria that express extracellular chaperonin proteins. In one embodiment, it relates to Mycobacterium tuberculosis (Mtb) detection and provides for novel probes for a specific and sensitive diagnostic test for Mycobacterium tuberculosis complex (TBC) that hybridize with the groEL-1 gene. Arrays comprising the novel probes immobilized on a support for hybridization analysis and methods for TBC detection using the probes are also provided.

### BACKGROUND OF THE INVENTION

Chaperonins, which are also called “heat shock proteins” or “stress proteins” are cell-signalling proteins that play a role in complex protein folding mechanisms. They are closely associated with essential cell survival strategies, and an trigger responses to a variety of stressful conditions, such as heat, cold, osmotic imbalance and exposure to toxins. As such, they are highly conserved throughout living organisms and are expressed on the surface of and excreted by both prokaryotic and eukaryotic cells.

There are two distinct families of chaperonins—the chaperonin 10 family and the chaperonin 60 family. They are both well characterized and highly related at the sequence level. In bacteria, the chaperonin 60 family, also referred to herein as “groEL”, and the chaperonin 10 family, also referred to herein as “groES”, have both been found to be essential for cell survival. Collectively, these two families can be referred to as the groE family of chaperoning.

In addition, when expressed by pathogenic bacteria such as Mycobacterium tuberculosis, chaperonins have been found to stimulate the release of cytokines from mammalian cells (Tabona, et al. J. Immunol 161:1414-1421 (1998). Other researchers have demonstrated that groEL from various pathogenic bacteria can also stimulate leukocytes, fibroblasts and epithelial cells to secrete cytokines. Even bacteria that normally colonize the oral cavity (e.g., Actinobacillus actinomycetemcomitans) and the intestines (e.g., Eschericia coli) have been implicated in the activation of bone resorption responses when these bacteria become “pathogenic” in immune compromised hosts such as the elderly.

Although the sequences of many chaperonin genes from various bacterial sources are known in the literature, most studies have focused on the proteins themselves as targets for immunodiagnostics and immunotherapeutics because chaperonins are highly immunogenic. In fact, the immunologic similarity of the chaperonin proteins from various sources would lead one to believe that antibodies specific for disease-related chaperonin targets might cross-react with chaperonins from normal cells. However, many researchers have found that chaperonin epitopes can serve as specific targets for immunodiagnostics and immunotherapeutics of bacterial infections such as Mycobacterium tuberculosis.

While most of the recent focus on chaperonins has been on their suitability as protein targets, the study of chaperonin genes as molecular targets for diagnostics and therapeutics of various disease states, and in particular pathogenic bacterial infections, has received much less attention. However, because the chaperonins are considered to be important bacterial virulance determinants, genetic targets in pathogenic bacteria associated with chaperonin protein expression are ideal candidates for new diagnostic and therapeutic approaches. Accordingly, the present invention focuses on such approaches, using the Mycobacterium tuberculosis groEL-1 gene as a model chaperonin genetic target.

Tuberculosis (TB) is one of the most deadly and common infectious diseases and claims three million lives a year worldwide. Although the disease is found mostly in developing countries, a growing number of cases are diagnosed in the industrialized countries. The causative agent of TB—Mycobacterium tuberculosis (Mtb)—is a slow-growing pathogen and is among the most recalcitrant in terms of clinical treatment. It is one of the Mycobacterium species in the TBC, which is a term used to refer to the species of Mycobacterium associated with TB. The other such species of TBC are M. bovis, M. bovis-BCG, M. africanum and M. microti. In addition, a deadly partnership is forged between Mtb and HIV. An increased susceptibility to TB is associated with early stages of the HIV infection, and TB in turn accelerates the progression to AIDS. The increased incidence of TB, especially in patients suffering with AIDS, makes early detection of Mtb crucial among these patients to provide for prompt treatment and cost-effective management to control the disease(s).

One reliable method of TB diagnosis is based on culturing M. tuberculosis from the clinical specimen and identifying it morphologically and biochemically. This usually takes anywhere from three to six weeks, during which time a patient may become seriously ill and infect other individuals. Therefore, a rapid test capable of reliably detecting the presence of M. tuberculosis is vital for the early detection and treatment.

M. tuberculosis H37Rv has been completely sequenced and its genome size is about 4.4 Mbp (Cole et al., Nature, 393:537-544 (1998)). The length of a segment #145/162 of this genome is 33,818 bp and is available as GenBank Accession #Z77165. The groEL-1 gene resides within this segment and encodes the heat shock/chaperonin 1 protein. GroEL1's coding sequence starts at nucleotide # 22,918 and ends at nucleotide # 24,537. The resulting sequence is 1,619 nucleotide long and encodes a 60 Kda protein.

Several molecular tests have been developed recently for the rapid detection and identification of M. tuberculosis. A commercial test is available from the Gen-Probe known as the Amplified Mycobacterium tuberculosis Direct Test (Abe et al., J. Clin. Microbiol., 31:3270-3274 (1993) and Miller et al., J. Clin. Microbiol., 32: 393-397 (1994)). This test involves amplification of M. tuberculosis 16S ribosomal RNA from respiratory specimens and uses a chemiluminescent probe to detect the amplified product with a reported sensitivity of about 91%. Other commercial tests based on ligase chain reaction (LCR by Abbott Laboratories), polymerase chain reaction (PCR by Roche Diagnostics Systems) and strand displacement amplification (SDA by Becton Dickinson) are discussed in a review by Forbes (Clin. Microbiol. Newsletter, 17:145-150 (1995)).

After the discovery of the IS6110 as being present only in TBC (Thierry et al., Nucleic Acids Research, 18:188 (1990)), rapid TB diagnostic strategies have been reported using IS6110 DNA sequence amplification methods (Kox et al., J. Clin. Microbiol., 32:672-678 (1994); and Miller et al., J. Clin. Microbiol., 32: 393-397 (1994)). Sandhu et al. (U.S. Pat. No. 5,731,150), Walker et al. (U.S. Pat. No. 5,470,723), Crawford et al. (U.S. Pat. Nos. 5,168,039 and 5,370,998) and Guesdon (European Patent EP 0,461,045) have described IS6110 based molecular detection of TBC using different primers and nucleic acid amplification methods. Although some of the methods are sensitive, some of them have problems due to nucleic acid contamination that give false positive results. In addition, some probes lack sufficient specificity to reproducibly detect TBC, and some of the detection methods are labor intensive and may not be cost-effective.

In contrast to the aforementioned genetic targets, the present invention focuses on chaperonin gene targets. In most bacterial species, a single operon (GroES/L) includes both the groES and groEL genes. The groES gene encodes a polypeptide of ˜10 kDa, while the groEL gene encodes a polypeptide of ˜60 kDa. However, in M. tuberculosis and M. leprae, cpn10 gene (a homolog to groES ) and cpn60-2/groEL-2 gene (a homolog to groEL or hsp65) are separate genes that are not expressed as a single operon. Kong et al. reported a second gene cpn60-1, another groEL homolog, located 98 bp downstream of the cpn10 gene in M. tuberculosis (Kong et al., Proc. Natl. Acad. Sci., USA, 90:2608-2612 (1993)). The product of cpn60-2 gene, HSP65/Cpn60-2, is a major target antigen for the vertebarte immune response to these pathogens (Young et al., Proc. Natl. Acad. Sci., USA, 85:4267-4270 (1988)).

Accordingly, the present invention involves the targeting of specific chaperonin gene targets from pathogenic bacteria to diagnose infections, particularly in mammalian hosts. In one embodiment, the invention includes the development of novel groEL-1 probes that can be used in any protocol of hybridization with or without nucleic acid amplification. These probes are highly specific and are capable of detecting TBC efficiently and reliably. They were selected based on their G+C content, length, Tm values and absence of hairpin secondary structure. In other embodiments, the present invention includes diagnostic methods and compositions based on chaperonin gene targets from any pathogenic bacteria that expresses chaperonin proteins associated with bacterial virulence.

### SUMMARY OF THE INVENTION

The present invention provides for molecular probes and uses thereof for a direct, specific and sensitive diagnostic test of Mycobacterium tuberculosis complex (TBC), which includes Mycobacterium tuberculosis (Mtb), a causative agent of tuberculosis. These TBC-specific probes hybridize with at least one groEL-1 gene sequence that is believed to be present only in TBC.

In one aspect, the present invention provides for an oligonucleotide probe for detecting TBC in a sample comprising a nucleotide sequence that hybridizes with a target nucleotide sequence, wherein the target nucleotide sequence is all or part of the groEL-1 gene (SEQ ID NO. 1; FIG. 1), or a complementary strand thereof, and wherein the probe has a G+C content from about 30 to 70%, a Tm value from about 55-90° C., a length of at least 8 nucleotides (if DNA and/or RNA, or 6 if PNA or other nucleic acid analog with a higher affinity than DNA or RNA) and does not contain any hairpin secondary structure. Preferably, the probe hybridizes with a target nucleotide sequence of a Mycobacterium tuberculosis groEL-1 gene under middle or high stringency.

In another aspect, the probe may comprise a nucleotide sequence that hybridizes under low stringency with a target comprising the nucleotide sequence shown below, or a complementary strand thereof.

(SEQ ID NO:2)

5′-TCAGTGCGCGTGCCCGTGGTGATGGTCGTGATCTTCTGCCTTGGCCGG
CTTGTCGACCACGACCGTCTCGGTGGTGAGTACCATCCGGGCAACCGATG
ACGCGTTCAACACCGCCGACCTAGTCACCTTGACCGGGTCGATGACGCCG
TCAGCGGCCAAGTCACCATAGCTCAGGGTGTTCACGTTCAGCCCATGCCC
GGCGGGTAGCTCGCTGACCTTGTTGACCACCACCGAGCCGTCCAAGCCAG
CGTTGGCGGCGATCCAGAACAACGGCGCGGCAAGGGCTTCGGAGAACAC
GTCGACACCGAGGACCTCGTCACCGGTCAGCGACGC-3′

In yet another aspect, the probe may comprise a nucleotide sequence that hybridizes under low stringency with a target comprising a nucleotide sequence, or a complementary strand thereof, selected from the group consisting of:

| | |
|---|---|
| 5′-TCAACACCGCCGACCTAGTCA-3′, | (SEQ ID NO:3) |
| 5′-CTTCGGAGAACACGTCGACA-3′, | (SEQ ID NO:4) |
| 5′-TCAGGGTGTTCACGTTCAGCCCAT-3′, | (SEQ ID NO:5) |
| 5′-GACGCGTTCAACACCGCCGACCTAGTCA-3′, | (SEQ ID NO:6) |
| 5′-CTTCGGAGAACACGTCGACAC-3′, | (SEQ ID NO:7) |
| and | |
| 5′-AACACGTCGACACCGAGGACCT-3′. | (SEQ ID NO:8) |

In still another aspect, the present invention provides for an oligonucleotide probe for detecting TBC in a sample comprising a nucleotide sequence having at least 90% identity with a sequnce selected from the group consisting of:

| | |
|---|---|
| 5′-TCAACACCGCCGACCTAGTCA-3′, | (SEQ ID NO:3) |
| 5′-CTTCGGAGAACACGTCGACA-3′, | (SEQ ID NO:4) |
| 5′-TCAGGGTGTTCACGTTCAGCCCAT-3′, | (SEQ ID NO:5) |
| 5′-GACGCGTTCAACACCGCCGACCTAGTCA-3′, | (SEQ ID NO:6) |
| 5′-CTTCGGAGAACACGTCGACAC-3′, | (SEQ ID NO:7) |
| 5′-AACACGTCGACACCGAGGACCT-3′, | (SEQ ID NO:8) |
| 3′-AGTTGTGGCGGCTGGATCAGT-5′, | (SEQ ID NO:9) |
| 3′-GAAGCCTCTTGTGCAGCTGT-5′, | (SEQ ID NO:10) |
| 3′-AGTCCCACAAGTGCAAGTCGGGTA-5′, | (SEQ ID NO:11) |
| 3′-CTGCGCAAGTTGTGGCGGCTGGATCAGT-5′, | (SEQ ID NO:12) |
| 3′-GAAGCCTCTTGTGCAGCTGTG-5′, | (SEQ ID NO:13) |
| and | |
| 3′-TTGTGCAGCTGTGGCTCCTGGA-5′. | (SEQ ID NO:14) |

Preferably, the oligonucleotide probe comprises a nucleotide sequence selected from the group consisting of:

| | |
|---|---|
| 5′-TCAACACCGCCGACCTAGTCA-3′, | (SEQ ID NO:3) |
| 5′-CTTCGGAGAACACGTCGACA-3′, | (SEQ ID NO:4) |
| 5′-TCAGGGTGTTCACGTTCAGCCCAT-3′, | (SEQ ID NO:5) |
| 5′-GACGCGTTCAACACCGCCGACCTAGTCA-3′, | (SEQ ID NO:6) |
| 5′-CTTCGGAGAACACGTCGACAC-3′, | (SEQ ID NO:7) |
| 5′-AACACGTCGACACCGAGGACCT-3′, | (SEQ ID NO:8) |
| 3′-AGTTGTGGCGGCTGGATCAGT-5′, | (SEQ ID NO:9) |
| 3′-GAAGCCTCTTGTGCAGCTGT-5′, | (SEQ ID NO:10) |
| 3′-AGTCCCACAAGTGCAAGTCGGGTA-5′, | (SEQ ID NO:11) |
| 3′-CTGCGCAAGTTGTGGCGGCTGGATCAGT-5′, | (SEQ ID NO:12) |
| 3′-GAAGCCTCTTGTGCAGCTGTG-5′, | (SEQ ID NO:13) |
| and | |
| 3′-TTGTGCAGCTGTGGCTCCTGGA-5′. | (SEQ ID NO:14) |

The probe may comprise DNA, RNA, PNA or a derivative thereof. It may also comprise both DNA and RNA or derivatives thereof, and may additionally be labeled. The label can be a chemical, an enzymatic, an immunogenic, a radioactive, a fluorescent, a luminescent or a FRET label.

The present invention further incudes an array of oligonucleotide probes immobilized on a support for detecting TBC, which array comprises a support suitable for use in nucleic acid hybridization having immobilized thereon a plurality of oligonucleotide probes, at least one of which is described above. The support may further comprise a surface that is selected from the group consisting of a silicon, a plastic, a glass, a ceramic, a rubber, and a polymer surface.

The present invention also provides a method for detecting TBC in a sample comprising the steps of: a) providing an oligonucleotide probe as described above; b) contacting the probe with a sample containing or suspected of containing a TBC target nucleotide sequence under conditions suitable for hybridization between the probe and the target nucleotide sequence; and c) assessing hybridization between the probe and the target nucleotide sequence to detect the TBC in said sample.

In any of the aforementioned methods, a plurality of samples can be assayed sequentially or simultaneously. The sample may be of human origin, but may also be sputum, urine, blood, tissue section, food, soil and water sample.

The methods described herein are useful for detecting any TBC.

In the aforementioned paragraphs, the detection of TBC is discussed as a model system for the practice of the present invention. However, another aspect of the present invention is a method for detecting pathogenic bacteria that excrete at least one chaperonin protein of the groE family, said method comprising the steps of:

a) providing an oligonucleotide probe comprising a nucleotide sequence that hybridizes under high stringency with a nucleotide sequence encoding a groE chaperonin protein, wherein the probe has a G+C content from about 30 to 70%, a Tm value from about 55 to 90° C., a length of at least 8 nucleotides and does not contain any hairpin secondary structure;

b) contacting said probe with a sample containing or suspected of containing said pathogenic bacteria under conditions suitable for hybridization between said probe and said nucleotide sequence encoding a groE chaperonin protein; and

c) assessing hybridization between said probe and said nucleotide sequence to detect said pathogenic bacteria in said sample.

Many such chaparonin-encoding gene sequences are known, as are methods for identifying subsequences within these sequences that are unique for the particular taxonomic group of pathogenic bacteria being studied.

### BRIEF DESCRIPTION OF THE DRAWINGS

FIG. 1 illustrates the nucleotide sequence of the gene encoding the groEL-1 protein (SEQ ID NO:1), which comprises nucleotides 22918 to 24537 of M. tuberculosis H37Rv, GenBank Accession Number Z77165. The subsequence (SEQ ID NO:2) representing a preferred binding targets are underlined.

FIG. 2 illustrates the specificity of AGT1051 (SEQ ID NO: 3), AGT1052 (SEQ ID NO: 4) and AGT1053 (SEQ ID NO: 5), which distinguishes Mtb DNA from M. chelonae DNA.

FIG. 3 illustrates the specificity of AGT1051 (SEQ ID NO: 3) for Mtb versus MOTT.

### DETAILED DESCRIPTION OF THE INVENTION

The present invention provides groEL-1 probes that are specific for TBS, e.g. Mycobacterium tuberculosis, and their use in diagnostic assays. The use of specific oligonucleotide sequences as probes in hybridization-based detection of infectious agents is becoming a valuable identification assay as an alternative to the problematic immuno-diagnostic and DNA amplification methods. The TBS-specific probes identified in this invention provide the basis for a complete “specimen to result” protocol using the probes with hybridization-based method and nucleic acid labeling method disclosed herein.

For clarity of disclosure, and not by way of limitation, the detailed description of the invention is divided into the subsections that follow.

### DEFINITIONS

Unless defined otherwise, all technical and scientific terms used herein have the same meaning as is commonly understood by one of ordinary skill in the art to which this invention belongs. If a definition set forth in this section is contrary to or otherwise inconsistent with a definition found in such incorporated references, the definition set forth in this section prevails over the definition that is incorporated herein by reference.

As used herein, “mycobacteria” refers to bacteria with unusual cell walls that are resistant to digestion, being waxy, very hydrophobic, and rich in lipid, especially esterified mycolic acids. Staining properties of mycobacteria differ from those of Gram negative and Gram positive organisms, being acid-fast. Many mycobacteria are intracellular parasites, causing serious diseases such as leprosy and tuberculosis. Mycobacteria cell wall has strong immuno-stimulating adjuvant properties due to muramyl dipeptide (MDP).

As used herein, “Mycobacterium tuberculosis” refers to the causing agent of the tuberculosis disease. Tuberculosis may affect almost any tissue or organ of the body, the most common seat of the disease being the lungs. The anatomical lesion of tuberculosis is the tubercle, which can undergo caseation necrosis. Local symptoms of tuberculosis vary according to the part affected. General symptoms of tuberculosis are those of sepsis, hectic fever, sweats, and emaciation.

As used herein, “chaperonin” refers to a member of the class of proteins also referred to as “heath shock” proteins, which includes groEL (a.k.a. chaperonin 60) and groES (also known as chaperonin 10), as well as fragments thereof.

As used herein, “pathogenic bacteria” refer to prokaryotes which are associated with one or more disease states and, for purposes of the present invention, express chaperonin proteins associated with bacterial virulence.

As used herein, “primer” refers to an oligonucleotide that hybridizes to a target sequence, typically to prime the nucleic acid in the amplification process.

As used herein, “probe” refers to an oligonucleotide that hybridizes to a target sequence, typically to facilitate its detection, but which also may serve as a primer. The term “target sequence” refers to a nucleic acid sequence to which the probe specifically binds. Unlike a primer that is used to prime the target nucleic acid in the amplification process, a probe need not be extended to amplify target sequence using a polymerase enzyme. However, it will be apparent to those skilled in the art that probes and primers are structurally similar or identical in many cases.

As used herein, “complementary” means that two nucleic acid sequences have at least 50% sequence identity. Preferably, the two nucleic acid sequences have at least 60%, 70%, 80%, 90%, 95%, 96%, 97%, 98%, 99% or 100% sequence identity. “Complementary” also means that two nucleic acid sequences can hybridize under low, middle and/or high stringency condition(s).

As used herein, “substantially complementary” means that two nucleic acid sequences have at least 90% sequence identity. Preferably, the two nucleic acid sequences have at least 95%, 96%, 97%, 98%, 99% or 100% sequence identity. Alternatively, “substantially complementary” means that two nucleic acid sequences can hybridize under high stringency condition(s).

As used herein, “two perfectly matched nucleotide sequences” refers to a nucleic acid duplex wherein the two nucleotide strands match according to the Watson-Crick basepair principle, e.g., A-T and C-G pairs in DNA:DNA duplex and A-U and C-G pairs in DNA:RNA or RNA:RNA duplex, and there is no deletions or additions in either of the sequences in the duplex.

As used herein: “stringency of hybridization” in determining percentage mismatch is as follows:

1) high stringency: 0.1×SSPE, 0.1% SDS, 65° C.;

2). medium stringency: 0.2×SSPE, 0.1% SDS, 50° C. (also referred to as moderate stringency); and

3) low stringency: 1.0×SSPE, 0.1% SDS, 50° C.

It is understood that equivalent stringencies may be achieved using alternative buffers, salts and temperatures (See generally, Ausubel (Ed.) Current Protocols in Molecular Biology, 2.9A. Southern Blotting, 2.9B. Dot and Slot Blotting of DNA and 2.10. Hybridization Analysis of DNA Blots, John Wiley & Sons, Inc. (2000)).

As used herein, “hairpin structure” refers to a nucleic acid that contains a double-stranded stem segment and a single-stranded loop segment wherein the two sides of the double-stranded stem segment are linked and separated by the single-stranded loop segment. The “hairpin structure” can also include 3′ and/or 5′ single-stranded region(s) extending from the double-stranded stem segment.

As used herein, “G+C content” refers to the percentage of the number of Gs (guanines) and Cs (cytoseines) in the nucleotide sequence, excluding poly T tails/spacers.

As used herein, a probe that “does not contain any hairpin secondary structure” means that the nucleotide sequence in the probe that is complementary to a target nucleotide sequence cannot form a hairpin structure within itself. However, the nucleotide sequence in the probe that is complementary to a target nucleotide sequence can be part of a nucleic acid that may form a hairpin structure under suitable conditions. For example, the nucleotide sequence complementary to a target nucleotide sequence can be located within a hairpin structure, or can be located at the junction of the loop and stem region of the hairpin structure.

As used herein, “melting temperature” (“Tm”) refers to the midpoint of the temperature range over which nucleic acid duplex, e.g., DNA:DNA, DNA:RNA and RNA:RNA, is denatured. The Tm of the probe herein means the Tm of the hybridized probe.

As used herein, “assessing” refers to quantitative and/or qualitative determination of the hybrid formed between the probe and the target nucleotide sequence, e.g., obtaining an absolute value for the amount or concentration of the hybrid, and also of obtaining an index, ratio, percentage, visual or other value indicative of the level of hybridization. Assessment may be direct or indirect, and the chemical species actually detected need not be the hybrid itself but may, for example, be a derivative thereof, reduction or disappearance of the probe and/or the target nucleotide sequence, or some further substance.

Probes for Detecting TBS, e.g. Mycobacterium tuberculosis

The present invention provides probes for detecting TBS that contain a nucleotide sequence that hybridizes with a target nucleotide sequence of a chaperonin protein, such as groEL-1. The probes can be in any suitable form. For example, the probe can comprise DNA, RNA, PNA or a derivative thereof. Alternatively, the probe can comprise both DNA and RNA or derivatives thereof. The probe can be single-stranded and be ready to be used in a hybridization analysis. Alternatively, the probe can be double-stranded and be denatured into single-stranded form prior to the hybridization analysis.

The oligonucleotide probes can be produced by any suitable method. For example, the probes can be chemically synthesized (See generally, Ausubel (Ed.) Current Protocols in Molecular Biology, 2.11. Synthesis and purification of oligonucleotides, John Wiley & Sons, Inc. (2000)), isolated from a natural source, produced by recombinant methods or a combination thereof. Synthetic oligonucleotides can be prepared by using the triester method of Matteucci et al., J. Am. Chem. Soc., 3: 3185-3191 (1981). Alternatively, automated synthesis maybe preferred, for example, on an Applied Biosynthesis DNA synthesizer using cyanoethyl phosphoramidate chemistry. Preferably, the probes are chemically synthesized.

Suitable bases for preparing the oligonucleotide probes of the present invention may be selected from naturally occurring nucleotide bases such as adenine, cytosine, guanine, uracil, and thymine. It may also be selected from non-naturally occurring or “synthetic” nucleotide bases such as 8-oxo-guanine, 6-mercaptoguanine, 4-acetylcytidine, 5-(carboxyhydroxyethyl)uridine, 2′-O-methylcytidine, 5-carboxymethylamino-methyl-2-thiouridine, 5-carboxymethylaminomethyl uridine, dihydrouridine, 2′-O-methylpseudouridine, beta-D-galactosylqueosine, 2′-O-methylguanosine, inosine, N⁶-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N⁶-methyladenosine, 7-methylguanosine, 5-methylaminomethyluridine, 5-methoxyaminomethyl-2-thiouridine, beta-D-mannosylqueosine, 5-methoxycarbonylmethyluridine, 5-methoxyuridine, 2-methylthio-N⁶-isopentenyladenosine, N-((9-.beta.-D-ribofuranosyl-2-methylthiopurine-6-yl)carbamoyl)threonine, N-((9-beta-D-ribofuranosylpurine-6-yl)N-methylcarbamoyl) threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-methyluridine, N-((9-beta-D-ribofuranosylpurine-6-yl) carbamoyl)threonine, 2′-O-methyl-5-methyluridine, 2′-O-methyluridine, wybutosine, and 3-(3-amino-3-carboxypropyl)uridine.

Likewise, chemical analogs of oligonucleotides (e.g., oligonucleotides in which the phosphodiester bonds have been modified, e.g., to the methylphosphonate, the phosphotriester, the phosphorothioate, the phosphorodithioate, or the phosphoramidate) may also be employed. Protection from degradation can be achieved by use of a “3′-cnd cap” strategy by which nuclease-resistant linkages are substituted for phosphodiester linkages at the 3′ end of the oligonucleotide (Shaw et al., Nucleic Acids Res. 19: 747-50 (1991)). Phosphoramidates, phosphorothioates, and methylphosphonate linkages all function adequately in this manner. More extensive modification of the phosphodiester backbone has been shown to impart stability and may allow for enhanced affinity and increased cellular permeation of oligonucleotides (Milligan et al., J. Med. Chem. 36: 1923-37 (1993)). Many different chemical strategies have been employed to replace the entire phosphodiester backbone with novel linkages. Backbone analogues include phosphorothioate, phosphorodithioate, methylphosphonate, phosphoramidate, boranophosphate, phosphotriester, formacetal, 3′-thioformacetal, 5′-thioformacetal, 5′-thioether, carbonate, 5′-N-carbamate, sulfate, sulfonate, sulfamate, sulfonamide, sulfone, sulfite, sulfoxide, sulfide, hydroxylamine, methylene (methylimino) (MMI) or methylencoxy (methylimino) (MOMI) linkages. Phosphorothioate and methylphosphonate-modifiedoligonucleotides are particularly preferred due to their availability through automated oligonucleotide synthesis. The oligonucleotide may be a “peptide nucleic acid” such as described by Nielsen et al., Science 254: 1497 (1991). The only requirement is that the oligonucleotide probe should possess a sequence at least a portion of which is capable of binding to a portion of the sequence of a target DNA molecule.

Hybridization probes can be of any suitable length. There is no lower or upper limits to the length of the probe, as long as the probe hybridizes to the target nucleic acid sequence(s) and functions effectively as a probe (e.g., facilitates detection). The probes of the present invention can be as short as 50, 40, 30, 20, 15, 10 or 8 nucleotides, or shorter. Likewise, the probes can be as long as 20, 40, 50, 60, 75, 100 or 200 nucleotides, or longer, e.g., to the full length of the sip sequence. Generally, the probes will have at least 8 nucleotides, preferably at least 14 nucleotides, more preferably at least 19 nucleotides, and will not contain any hairpin secondary structures. In specific embodiments, the probe can have a length of at least 30 nucleotides or at least 50 nucleotides. If there is to be complete complementarity, e.g., if the target strand contains a sequence identical to that of the probe, the duplex will be relatively stable under even stringent conditions and the probes may be short, e.g., in the range of about 10-30 base pairs. If some degree of mismatch is expected in the probe, e.g., if it is suspected that the probe would hybridize to a variant region, or to a group of sequences such as all species within a specific genus, e.g., Mycobacterium species, the probe may be of greater length (e.g., 15-40 bases) to balance the effect of the mismatch(es).

The probe should have a G+C content ranging from about 30% to about 70%. Preferably, the probe has G+C content ranging from about 55% to about 65%. The probe should have a Tm value ranging from about 55° C. to about 90° C., preferably from about 65° C. to about 85° C.

The probes used in the present invention are selected to be “substantially complementary” to the different strands of each specific sequence to be hybridized. The probes need not reflect the exact sequence of the template, but must be sufficiently complementary to hybridize selectively with their respective strands. Non-complementary bases or longer sequences can be interspersed into the probe, provided that the probe retains sufficient complementarity with the sequence of one of the strands to be hybridized to form a duplex structure which can be detected. The non-complementary nucleotide sequences of the probes may include restriction enzyme sites. Appending a restriction enzyme site to the end(s) of the target sequence is particularly helpful for subsequent cloning of the target sequence.

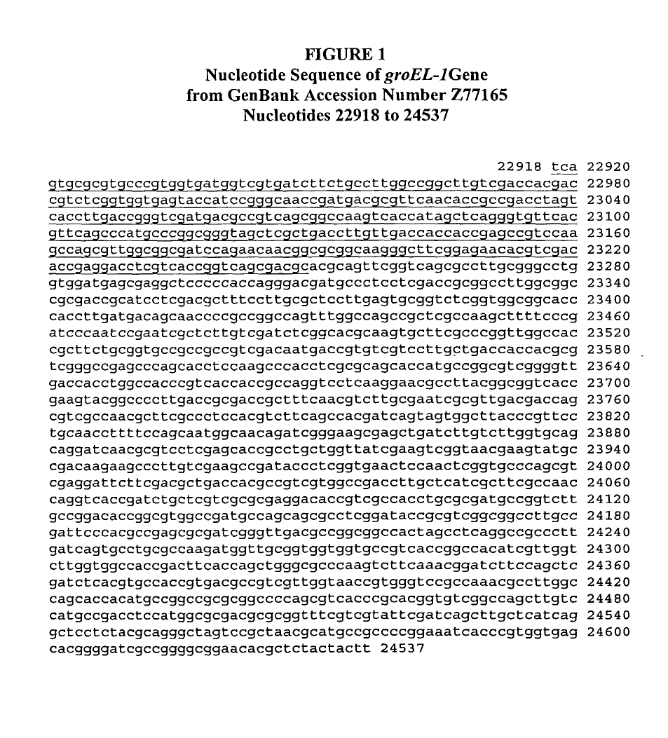

The probe should also be specific for a pathogenic bacteria, e.g. TBC. In other words, it should be capable of differentiating TBC from MOTT. Any suitable contiguous nucleotide sequence that is at least 8 nucleotides in length, or a complementary strand thereof, within a chaperonin encoding sequence specific for the pathogenic bacteria, such as the groEL-1 sequence (SEQ ID NO:1) can be used as a target nucleotide sequence. In addition, there are certain subsequences within the groEL-1 gene sequence that are preferred target sequences. One such sequence is shown in underlined text in FIG. 1, and is as follows:

(SEQ ID NO:2)

5′-TCAGTGCGCGTGCCCGTGGTGATGGTCGTGATCTTCTGCCTTGGCCGG
CTTGTCGACCACGACCGTCTCGGTGGTGAGTACCATCCGGGCAACCGATG
ACGCGTTCAACACCGCCGACCTAGTCACCTTGACCGGGTCGATGACGCCG
TCAGCGGCCAAGTCACCATAGCTCAGGGTGTTCACGTTCAGCCCATGCCC
GGCGGGTAGCTCGCTGACCTTGTTGACCACCACCGAGCCGTCCAAGCCAG
CGTTGGCGGCGATCCAGAACAACGGCGCGGCAAGGGCTTCGGAGAACAC
GTCGACACCGAGGACCTCGTCACCGGTCAGCGACGC-3′

Typically, the probes of the present invention hybridize to consecutive nucleotides of the groEL-1 sequence under stringent conditions, as defined elsewhere. Alternatively stated, probes of the present invention will be at least 75%, 80%, 85%, 90% or even 95% homologous or more with consecutive nucleotides within the sip sequence, or complementary sequences thereof.

Preferably, the present invention provides for an oligonucleotide probe for detecting TBS in a sample comprising a nucleotide sequence that hybridizes with a target nucleotide sequence, wherein the target nucleotide sequence is all or part of the groEL-1 gene, or a complementary strand thereof, and wherein the probe has a G+C content from about 30 to 70%, a Tm value from about 55 to 90° C., a length of at least 8 nucleotides (if DNA and/or RNA, or 6 if PNA or other nucleic acid analog with a higher affinity than DNA or RNA) and does not contain any hairpin secondary structure. Preferably, the probe hybridizes with a target nucleotide sequence of a Mycobacterium tuberculosis groEL-1 gene under middle or high stringency.

The sequences of exemplary probes are:

(SEQ ID NO:3; a.k.a. AGT1051)

5′-TCAACACCGCCGACCTAGTCA-3′,

(SEQ ID NO:4; a.k.a. AGT1052)

5′-CTTCGGAGAACACGTCGACA-3′,

(SEQ ID NO:5; a.k.a. AGT1053)

5′-TCAGGGTGTTCACGTTCAGCCCAT-3′,

(SEQ ID NO:6; a.k.a. Tb-groEL1-01)

5′-GACGCGTTCAACACCGCCGACCTAGTCA-3′,

(SEQ ID NO:7; a.k.a. Tb-groEL1-03)

5′-CTTCGGAGAACACGTCGACAC-3′,
and

(SEQ ID NO:8; a.k.a. TB-groEL1-06)

5′-AACACGTCGACACCGAGGACCT-3′.

Immobilization of Probes

The present invention provides, e.g., a method for detecting TBC in a sample comprising the steps of: a) providing an oligonucleotide probe as described above; b) contacting the probe provided in step a) with a sample suspected of containing a TBC target nucleotide sequence under conditions suitable for hybridization between the probe and the target nucleotide sequence; and c) assessing hybridization between the probe and the target nucleotide sequence to detect TBC in the sample. The present invention also provides an array of immobilized oligonucleotide probes for detecting (TBC).

The present method can be used in solution. Preferably, it is conducted in chip format, e.g., by using the probe(s) immobilized on a solid support.

The probes can be immobilized on any suitable surface, preferably, a solid support, such as silicon, plastic, glass, ceramic, rubber, or polymer surface. The probe may also be immobilized in a 3-dimensional porous gel substrate, e.g., Packard HydroGel chip (Broude et al., Nucleic Acids Res., 29(19):E92 (2001)).

For an array-based assay, the probes are preferably immobilized to a solid support such as a “biochip”. The solid support may be biological, nonbiological, organic, inorganic, or a combination of any of these, existing as particles, strands, precipitates, gels, sheets, tubing, spheres, containers, capillaries, pads, slices, films, plates, slides, etc.

A microarray biochip containing a library of probes can be prepared by a number of well known approaches including, for example, light-directed methods, such as VLSIPS™ described in U.S. Pat. Nos. 5,143,854, 5,384,261 or 5,561,071; bead based methods such as described in U.S. Pat. No. 5,541,061; and pin based methods such as detailed in U.S. Pat. No. 5,288,514. U.S. Pat. No. 5,556,752, which details the preparation of a library of different double stranded probes as a microarray using the VLSIPS™, is also suitable for preparing a library of hairpin probes in a microarray.

Flow channel methods, such as described in U.S. Pat. Nos. 5,677,195 and 5,384,261, can be used to prepare a microarray biochip having a variety of different probes. In this case, certain activated regions of the substrate are mechanically separated from other regions when the probes are delivered through a flow channel to the support. A detailed description of the flow channel method can be found in U.S. Pat. No. 5,556,752, including the use of protective coating wetting facilitators to enhance the directed channeling of liquids though designated flow paths.

Spotting methods also can be used to prepare a microarray biochip with a variety of probes immobilized thereon. In this case, reactants are delivered by directly depositing relatively small quantities in selected regions of the support. In some steps, of course, the entire support surface can be sprayed or otherwise coated with a particular solution. In particular formats, a dispenser moves from region to region, depositing only as much probe or other reagent as necessary at each stop. Typical dispensers include micropipettes, nanopippettes, ink-jet type cartridges and pins to deliver the probe containing solution or other fluid to the support and, optionally, a robotic system to control the position of these delivery devices with respect to the support. In other formats, the dispenser includes a series of tubes or multiple well trays, a manifold, and an array of delivery devices so that various reagents can be delivered to the reaction regions simultaneously. Spotting methods are well known in the art and include, for example, those described in U.S. Pat. Nos. 5,288,514, 5,312,233 and 6,024,138. In some cases, a combination of flow channels and “spotting” on predefined regions of the support also can be used to prepare microarray biochips with immobilized probes.

A solid support for immobilizing probes is preferably flat, but may take on alternative surface configurations. For example, the solid support may contain raised or depressed regions on which probe synthesis takes place or where probes are attached. In some embodiments, the solid support can be chosen to provide appropriate light-absorbing characteristics. For example, the support may be a polymerized Langmuir Blodgett film, glass or functionalized glass, Si, Ge, GaAs, GaP, SiO₂, SiN₄, modified silicon, or any one of a variety of gels or polymers such as (poly)tetrafluoroethylene, (poly)vinylidendifluoride, polystyrene, polycarbonate, or combinations thereof. Other suitable solid support materials will be readily apparent to those of skill in the art.

The surface of the solid support can contain reactive groups, which include carboxyl, amino, hydroxyl, thiol, or the like, suitable for conjugating to a reactive group associated with an oligonucleotide or a nucleic acid. Preferably, the surface is optically transparent and will have surface Si—OH functionalities, such as those found on silica surfaces.

The probes can be attached to the support by chemical or physical means such as through ionic, covalent or other forces well known in the art. Immobilization of nucleic acids and oligonucleotides can be achieved by any means well known in the art (see, e.g., Dattagupta et al., Analytical Biochemistry, 177:85-89(1989); Saiki et al., Proc. Natl. Acad. Sci. USA, 86:6230-6234(1989); and Gravitt et al., J. Clin. Micro., 36:3020-3027(1998)).

The probes can be attached to a support by means of a spacer molecule, e.g., as described in U.S. Pat. No. 5,556,752 to Lockhart et al., to provide space between the double stranded portion of the probe as may be helpful in hybridization assays. A spacer molecule typically comprises between 6-50 atoms in length and includes a surface attaching portion that attaches to the support. Attachment to the support can be accomplished by carbon-carbon bonds using, for example, supports having (poly)trifluorochloroethylene surfaces, or preferably, by siloxane bonds (using, for example, glass or silicon oxide as the solid support). Siloxane bonding can be formed by reacting the support with trichlorosilyl or trialkoxysilyl groups of the spacer. Aminoalkylsilanes and hydroxyalkylsilanes, bis(2-hydroxyethyl)-aminopropyltriethoxysilane, 2-hydroxyethylaminopropyltriethoxysilane, aminopropyltriethoxysilane or hydroxypropyltriethoxysilane are useful are surface attaching groups.

The spacer can also include an extended portion or longer chain portion that is attached to the surface-attaching portion of the probe. For example, amines, hydroxyl, thiol, and carboxyl groups are suitable for attaching the extended portion of the spacer to the surface-attaching portion. The extended portion of the spacer can be any of a variety of molecules which are inert to any subsequent conditions for polymer synthesis. These longer chain portions will typically be aryl acetylene, ethylene glycol oligomers containing 2-14 monomer units, diamines, diacids, amino acids, peptides, or combinations thereof.

In some embodiments, the extended portion of the spacer is a polynucleotide or the entire spacer can be a polynucleotide. The extended portion of the spacer also can be constructed of polyethyleneglycols, polynucleotides, alkylene, polyalcohol, polyester, polyamine, polyphosphodiester and combinations thereof. Additionally, for use in synthesis of probes, the spacer can have a protecting group attached to a functional group (e.g., hydroxyl, amino or carboxylic acid) on the distal or terminal end of the spacer (opposite the solid support). After deprotection and coupling, the distal end can be covalently bound to an oligomer or probe.

The present method can be used to analyze a single sample with a single probe at a time. Preferably, the method is conducted in high-throughput format. For example, a plurality of samples can be analyzed with a single probe simultaneously, or a single sample can be analyzed using a plurality of probes simultaneously. More preferably, a plurality of samples can be analyzed using a plurality of probes simultaneously.

In a specific embodiment, the probe or the TBC target nucleotide sequence is immobilized on a solid support. In another specific embodiment, a plurality of the probes immobilized on a solid support is used. In still another specific embodiment, a plurality of samples is assayed. Preferably, the plurality of samples is assayed simultaneously.

Hybridization Conditions

Hybridization can be carried out under any suitable technique known in the art. It will be apparent to those skilled in the art that hybridization conditions can be altered to increase or decrease the degree of hybridization, the level of specificity of the hybridization, and the background level of non-specific binding (e.g., by altering hybridization or wash salt concentrations or temperatures). The hybridization between the probe and the target nucleotide sequence can be carried out under any suitable stringencies, including high, middle or low stringency. Typically, hybridizations will be performed under conditions of high stringency.

The hybridization can be carried out at any suitable temperature. For example, if the present probe is part of a hairpin structure, the oligonucleotide probe and the target nucleotide sequence can be contacted at a temperature from about 420 C. to about 90° C. Preferably, the oligonucleotide probe and the target nucleotide sequence can be contacted at a temperature from about 40° C. to about 90° C., preferably from about 60° C. to about 75° C.

In addition, the hybridization can be carried out for any suitable period of time. For example, if the present probe is part of a hairpin structure as disclosed in co-owned PCT Patent Application No. WO 02/106531, the oligonucleotide probe and the target nucleotide sequence can be contacted for a time from about 1 minute to about 60 minutes. Preferably, the oligonucleotide probe and the target nucleotide sequence can be contacted for a time from about 15 minutes to about 30 minutes.

Hybridization between the probe and target nucleic acids can be homogenous, e.g., typical conditions used in molecular beacons (Tyagi S. et al., Nature Biotechnology, 14:303-308 (1996); and U.S. Pat. No. 6,150,097) and in hybridization protection assay (Gen-Probe, Inc.) (U.S. Pat. No. 6,004,745), or heterogeneous (typical conditions used in different type of nitrocellulose based hybridization and those used in magnetic bead based hybridization).

The target polynucleotide sequence may be detected by hybridization with an oligonucleotide probe that forms a stable hybrid with that of the target sequence under high to low stringency hybridization and wash conditions. An advantage of detection by hybridization is that, depending on the probes used, additional specificity is possible. If it is expected that the probes will be completely complementary (e.g., about 99% or greater) to the target sequence, high stringency conditions will be used. If some mismatching is expected, for example, if variant strains are expected with the result that the probe will not be completely complementary, the stringency of hybridization may be lessened. However, conditions are selected to minimize or eliminate nonspecific hybridization.

Species-specific hybridization of a target sequence refers, for example, to hybridization of a target sequence in Mycobacterium tuberculosis, but little or no hybridization to sequences from non-Mtb. The probes that are disclosed here hybridize to sip nucleic acids. Typically, the probes of the present invention hybridize to consecutive nucleotides of the target sequences, such as the groEL-1 sequence under stringent conditions, as defined below. Alternatively stated, probes of the present invention will be at least 75%, 80%, 85%, 90% or even 95% homologous or more with consecutive nucleotides within the groEL-1 sequence (or complementary strands thereof), in particular probes AGT 1051, AGT 1052, AGT1053, TB-groEL-01, TB-groEL1-03 and TB-groEL1-06, and complementary sequences thereof. As nucleic acids do not require complete homology to hybridize, it will be apparent to those skilled in the art that the probe sequences specifically disclosed herein may be modified so as to be substantially homologous to the probe sequences disclosed herein without loss of utility as TBC probes.

Stringency requirements can be modified to alter target specificity as described. For example, where TBC is to be detected, it is well within the scope of the invention for those of ordinary skill in the art to modify the stringency conditions described above and cause other non-TBC to be excluded or included as targets. The new groEL-1 probes provided herein give particular hybridization characteristics as desired.

Conditions those affect hybridization and those select against nonspecific hybridization are known in the art (Molecular Cloning: A Laboratory Manual, second edition, J. Sambrook, E. Fritsch, T. Maniatis, Cold Spring Harbor Laboratory Press, 1989). Generally, lower salt concentration and higher temperature increase the stringency of hybridization. For example, in general, stringent hybridization conditions include incubation in solutions that contain approximately 0.1×SSC, 0.1% SDS, at about 65° C. incubation/wash temperature. Middle stringent conditions are incubation in solutions that contain approximately 1-2×SSC, 0.1% SDS and about 50° C.-65° C. incubation/wash temperature. The low stringency conditions are 2×SSC and about 30° C.-50° C.

An alternate method of hybridization and washing is first to carry out a low stringency hybridization (5×SSPE, 0.5% SDS) followed by a high stringency wash in the presence of 3M tetramethyl-ammonium chloride (TMAC). The effect of the TMAC is to equalize the relative binding of A-T and G-C base pairs so that the efficiency of hybridization at a given temperature corresponds more closely to the length of the polynucleotide. Using TMAC, it is possible to vary the temperature of the wash to achieve the level of stringency desired (Wood et al., Proc. Natl. Acad. Sci. USA, 82:1585-1588 (1985)).

A hybridization solution may contain 25% formamide, 5×SSC, 5× Denhardt's solution, 100 μg/ml of single stranded DNA, 5% dextran sulfate, or other reagents known to be useful for probe hybridization.

Detection of the Hybrid

Detection of hybridization between the probe and the GBS nucleic acids can be carried out by any method known in the art, e.g., labeling the probe, the secondary probe, the target nucleic acids or some combination thereof, and are suitable for purposes of the present invention. Alternatively, the hybrid may be detected by mass spectroscopy in the absence of detectable label (e.g., U.S. Pat. No. 6,300,076).

The detectable label is a moiety that can be detected either directly or indirectly after the hybridization. In other words, a detectable label has a measurable physical property (e.g., fluorescence or absorbance) or is participant in an enzyme reaction. Using direct labeling, the target nucleotide sequence or the probe is labeled, and the formation of the hybrid is assessed by detecting the label in the hybrid. Using indirect labeling, a secondary probe is labeled, and the formation of the hybrid is assessed by the detection of a secondary hybrid formed between the secondary probe and the original hybrid.

Methods of labeling probes or nucleic acids are well known in the art. Suitable labels include fluorophores, chromophores, luminophores, radioactive isotopes, electron dense reagents, FRET(fluorescence resonance energy transfer), enzymes and ligands having specific binding partners. Particularly useful labels are enzymatically active groups such as enzymes (Wisdom, Clin. Chem., 22:1243 (1976)); enzyme substrates (British Pat. No. 1,548,741); coenzymes (U.S. Pat. Nos. 4,230,797 and 4,238,565) and enzyme inhibitors (U.S. Pat. No. 4,134,792); fluorophores (Soini and Hemmila, Clin. Chem., 25:353 (1979)); chromophores including phycobiliproteins, luminescers such as chemiluminescers and bioluminescers (Gorus and Schram, Clin. Chem., 25:512 (1979) and ibid, 1531); specifically bindable ligands, e.g., protein binding ligands; antigens; and residues comprising radioisotopes such as ³H, ³⁵S, ³²P, ¹²⁵I, and ¹⁴C. Such labels are detected on the basis of their own physical properties (e.g., fluorescers, chromophores and radioisotopes) or their reactive or binding properties (e.g., antibodies, enzymes, substrates, coenzymes and inhibitors). Ligand labels are also useful for solid phase capture of the oligonucleotide probe (e.g., capture probes). Exemplary labels include biotin (detectable by binding to labeled avidin or streptavidin) and enzymes, such as horseradish peroxidase or alkaline phosphatase (detectable by addition of enzyme substrates to produce a colored reaction product).

For example, a radioisotope-labeled probe or target nucleic acid can be detected by autoradiography. Alternatively, the probe or the target nucleic acid labeled with a fluorescent moiety can detected by fluorimetry, as is known in the art. A hapten or ligand (e.g., biotin) labeled nucleic acid can be detected by adding an antibody or an antibody pigment to the hapten or a protein that binds the labeled ligand (e.g., avidin).

As a further alternative, the probe or nucleic acid may be labeled with a moiety that requires additional reagents to detect the hybridization. If the label is an enzyme, the labeled nucleic acid, e.g., DNA, is ultimately placed in a suitable medium to determine the extent of catalysis. For example, a cofactor-labeled nucleic acid can be detected by adding the enzyme for which the label is a cofactor and a substrate for the enzyme. Thus, if the enzyme is a phosphatase, the medium can contain nitrophenyl phosphate and one can monitor the amount of nitrophenol generated by observing the color. If the enzyme is a beta-galactosidase, the medium can contain o-nitro-phenyl-D-galacto-pyranoside, which also liberates nitrophenol. Exemplary examples of the latter include, but are not limited to, beta-galactosidase, alkaline phosphatase, papain and peroxidase. For in situ hybridization studies, the final product of the substrate is preferably water insoluble. Other labels, e.g., dyes, will be evident to one having ordinary skill in the art.

The label can be linked directly to the DNA binding ligand, e.g., acridine dyes, phenanthridines, phenazines, furocoumarins, phenothiazines and quinolines, by direct chemical linkage such as involving covalent bonds, or by indirect linkage such as by the incorporation of the label in a microcapsule or liposome, which in turn is linked to the binding ligand. Methods by which the label is linked to a DNA binding ligand such as an intercalator compound are well known in the art and any convenient method can be used. Representative intercalating agents include mono-or bis-azido aminoalkyl methidium or ethidium compounds, ethidium monoazide ethidium diazide, ethidium dimer azide (Mitchell et al., J. Am. Chem. Soc., 104:4265 (1982)), 4-azido-7-chloroquinoline, 2-azidofluorene, 4′-aminomethyl-4,5′-dimethylangelicin, 4′-aminomethyl-trioxsalen (4′aminomethyl-4,5′,8-trimethyl-psoralen), 3-carboxy-5- or -8-amino- or -hydroxy-psoralen. A specific nucleic acid binding azido compound has been described by Forster et al., Nucleic Acid Res., 13:745 (1985). Other useful photoreactable intercalators are the furocoumarins which form (2+2) cycloadducts with pyrimidine residues. Alkylating agents also can be used as the DNA binding ligand, including, for example, bis-chloroethylamines and epoxides or aziridines, e.g., aflatoxins, polycyclic hydrocarbon epoxides, mitomycin and norphillin A. Particularly useful photoreactive forms of intercalating agents are the azidointercalators. Their reactive nitrenes are readily generated at long wavelength ultraviolet or visible light and the nitrenes of arylamides prefer insertion reactions over their rearrangement products (White et al., Meth. Enzymol., 46:644 (1977)).

The probe may also be modified for use in a specific format such as the addition of 10-100 T residues for reverse dot blot or the conjugation to bovine serum albumin or immobilization onto magnetic beads.

When detecting hybridization by an indirect detection method, a detectably labeled second probe(s) can be added after initial hybridization between the probe and the target or during hybridization of the probe and the target. Optionally, the hybridization conditions may be modified after addition of the secondary probe. After hybridization, unhybridized secondary probe can be separated from the initial probe, for example, by washing if the initial probe is immobilized on a solid support. In the case of a solid support, detection of label bound to locations on the support indicates hybridization of a target nucleotide sequence in the sample to the probe.

The detectably labeled secondary probe can be a specific probe. Alternatively, the detectably labeled probe can be a degenerate probe, e.g., a mixture of sequences such as whole genomic DNA essentially as described in U.S. Pat. No. 5,348,855. In the latter case, labeling can be accomplished with intercalating dyes if the secondary probe contains double stranded DNA. Preferred DNA-binding ligands are intercalator compounds such as those described above.

A secondary probe also can be a library of random nucleotide probe sequences. The length of a secondary probe should be decided in view of the length and composition of the primary probe or the target nucleotide sequence on the solid support that is to be detected by the secondary probe. Such a probe library is preferably provided with a 3′ or 5′ end labeled with photoactivatable reagent and the other end loaded with a detection reagent such as a fluorophore, enzyme, dye, luminophore, or other detectably known moiety.

The particular sequence used in making the labeled nucleic acid can be varied. Thus, for example, an amino-substituted psoralen can first be photochemically coupled with a nucleic acid, the product having pendant amino groups by which it can be coupled to the label, e.g., labeling is carried out by photochemically reacting a DNA binding ligand with the nucleic acid in the test sample. Alternatively, the psoralen can first be coupled to a label such as an enzyme and then to the nucleic acid.

Advantageously, the DNA binding ligand is first combined with label chemically and thereafter combined with the nucleic acid probe. For example, since biotin carries a carboxyl group, it can be combined with a furocoumarin by way of amide or ester formation without interfering with the photochemical reactivity of the furocoumarin or the biological activity of the biotin. Aminomethylangelicin, psoralen and phenanthridium derivatives can similarly be linked to a label, as can phenanthridium halides and derivatives thereof such as aminopropyl methidium chloride (Hertzberg et al, J. Amer. Chem. Soc., 104:313 (1982)). Alternatively, a bifunctional reagent such as dithiobis succinimidyl propionate or 1,4-butanediol diglycidyl ether can be used directly to couple the DNA binding ligand to the label where the reactants have alkyl amino residues, again in a known manner with regard to solvents, proportions and reaction conditions. Certain bifunctional reagents, possibly glutaraldehyde may not be suitable because, while they couple, they may modify nucleic acid and thus interfere with the assay. Routine precautions can be taken to prevent such difficulties.

Also advantageously, the DNA binding ligand can be linked to the label by a spacer, which includes a chain of up to about 40 atoms, preferably about 2 to 20 atoms, including, but not limited to, carbon, oxygen, nitrogen and sulfur. Such spacer can be the polyfunctional radical of a member including, but not limited to, peptide, hydrocarbon, polyalcohol, polyether, polyamine, polyimine and carbohydrate, e.g., -glycyl-glycyl-glycyl- or other oligopeptide, carbonyl dipeptides, and omega-amino-alkane-carbonyl radical or the like. Sugar, polyethylene oxide radicals, glyceryl, pentaerythritol, and like radicals also can serve as spacers. Spacers can be directly linked to the nucleic acid-binding ligand and/or the label, or the linkages may include a divalent radical of a coupler such as dithiobis succinimidyl propionate, 1,4-butanediol diglycidyl ether, a diisocyanate, carbodiimide, glyoxal, glutaraldehyde, or the like.

Secondary probe for indirect detection of hybridization can be also detected by energy transfer such as in the “beacon probe” method described by Tyagi and Kramer, Nature Biotech., 14:303-309 (1996) or U.S. Pat. Nos. 5,119,801 and 5,312,728 to Lizardi et al. Any FRET detection system known in the art can be used in the present method. For example, the AlphaScreen™ system can be used. AlphaScreen technology is an “Amplified Luminescent Proximity Homogeneous Assay” method. Upon illumination with laser light at 680 nm, a photosensitizer in the donor bead converts ambient oxygen to singlet-state oxygen. The excited singlet-state oxygen molecules diffuse approximately 250 nm (one bead diameter) before rapidly decaying. If the acceptor bead is in close proximity of the donor bead, by virtue of a biological interaction, the singlet-state oxygen molecules reacts with chemiluminescent groups in the acceptor beads, which immediately transfer energy to fluorescent acceptors in the same bead. These fluorescent acceptors shift the emission wavelength to 520-620 nm. The whole reaction has a 0.3 second half-life of decay, so measurement can take place in time-resolved mode. Other exemplary FRET donor/acceptor pairs include fluorescein (donor) and tetramethylrhodamine (acceptor) with an effective distance of 55 Å; IAEDANS (donor) and fluorescein (acceptor) with an effective distance of 46 Å; and fluorescein (donor) and QSY-7 dye (acceptor) with an effective distance of 61 Å (Molecular Probes).

Quantitative assays for nucleic acid detection also can be performed according to the present invention. The amount of secondary probe bound to a microarray spot can be measured and can be related to the amount of nucleic acid target which is in the sample. Dilutions of the sample can be used along with controls containing known amount of the target nucleic acid. The precise conditions for performing these steps will be apparent to one skilled in the art. In microarray analysis, the detectable label can be visualized or assessed by placing the probe array next to x-ray film or phosphoimagers to identify the sites where the probe has bound. Fluorescence can be detected by way of a charge-coupled device (CCD) or laser scanning.

Test Samples

Any suitable samples, including samples of human, animal, or environmental (e.g., soil or water) origin, can be analyzed using the present method. Test samples can include body fluids, such as urine, blood, semen, cerebrospinal fluid, pus, amniotic fluid, tears, or semisolid or fluid discharge (e.g., sputum, saliva, lung aspirate, vaginal or urethral discharge), stool or solid tissue samples (e.g., a biopsy or chorionic villi specimens). Test samples also include samples collected with swabs from the skin, genitalia, or throat.

Test samples can be processed to isolate nucleic acid by a variety of means well known in the art. See generally, Ausubel (Ed.) Current Protocols in Molecular Biology, 2. Preparation and Analysis of DNA and 4. Preparation and Analysis of RNA, John Wiley & Sons, Inc. (2000). It will be apparent to those skilled in the art that target nucleic acids can be RNA or DNA that may be in form of direct sample or purified nucleic acid or amplicons.

Purified nucleic acids can be extracted from the aforementioned samples and may be measured spectraphotometrically or by other instrument for the purity. For those skilled in the art of nucleic acid amplification, amplicons are obtained as end products by various amplification methods such as PCR (Polymerase Chain Reaction, U.S. Pat. Nos. 4,683,195, 4,683,202, 4,800,159 and 4,965,188), NASBA (Nucleic Acid Sequence Based Amplification, U.S. Pat. No. 5,130,238), TMA (Transcription Mediated Amplification) (Kwoh et al., Proc. Natl. Acad. Sci., USA, 86:1173-1177 (1989)), SDA (Strand Displacement Amplification, described by Walker et al., U.S. Pat. No. 5,270,184), tSDA (thermophilic Strand Displacement Amplification (U.S. Pat. No. 5,648,211 and Euro. Patent No. EP 0 684315), SSSR (Self-Sustained Sequence Replication) (U.S. Pat. No. 6,156,508).

In a specific embodiment, a sample of human origin-is assayed. In yet another specific embodiment, a sputum, urine, blood, tissue section, food, soil, or water sample is assayed.

Kits

The present probes can be packaged in a kit format, preferably with an instruction for using the probes to detect GBS. The components of the kit are packaged together in a common container, typically including written instructions for performing selected specific embodiments of the methods disclosed herein. Components for detection methods, as described herein, may optionally be included in the kit, for example, a second probe, and/or reagents and means for carrying out label detection (e.g., radiolabel, enzyme substrates, antibodies, etc., and the like).

The compositions and methods described herein can be used to detect any pathogenic bacteria, such as Mycobacterium tuberculosis and other prokaryotes known to experess chaperonin proteins.

### EXAMPLES

### Example 1

### Selecting Probes for the Detection of TBC

Probes were designed that would hybridize with the TBC groEL-1 sequence (SEQ ID NO:1) with high specificity and sensitivity in a way to avoid false positive results. An exemplary sub-regions of the groEL-1 gene that appeared to be more conserved among the TBC was identified as a potential probe site, and is given by SEQ ID NO:2. All nucleic acid databases in GenBank were search using the BLASTN 2.1.3 program, available online at the NCBI web site. The databases were searched using the GenBank Accession No.Z77165 as a query sequence with either multiple or pair wise alignments, and with Expect Value at 10 (relatively more permissive) initially.

Searches were carried out at Expect Value 1 (relatively less permissive). The searches produced a region with significant Blast Hits to explore for designing the probes. This was the region between nucleotides 23,000 and 23,250 (SEQ ID NO:2)/Several scans of each of these regions were analyzed and the consensus regions of the Blast Hits for the TBS were studied.

After an intensive analysis for hybridization characteristics, secondary hairpin structure and thermal profiles, several probes were selected from different sub-regions that were highly specific for sip genes. The sequences of these potential probes were then used as query sequences and compared to all nucleic acid databases in GenBank. The probes having significant identity with sequences of other genes in different organisms were eliminated and six probes were analyzed for their base composition, G+C content, thermal stability, hybridization characteristics and absence of hairpin structure formation.

Three of these probes (AGT1051, 1052 and 1053) were synthesized. Referring to FIG. 1, AGT1051 is an oligonucleotide sequence starting at nucleotide # 23,022 and ending at nucleotide # 23,042; AGT1052 is an oligonucleotide sequence starting at nucleotide # 23,202 and ending at nucleotide # 23,221; and AGT1053 is an oligonucleotide sequence starting at nucleotide # 23,088 and ending at nucleotide # 23,111. They are as follows:

(SEQ ID NO:3; a.k.a. AGT1051)

5′-TCAACACCGCCGACCTAGTCA-3′,

(SEQ ID NO:4; a.k.a. AGT1052),

5′-CTTCGGAGAACACGTCGACA-3′,

(SEQ ID NO:5; a.k.a. AGT1053)

5′-TCAGGGTGTTCACGTTCAGCCCAT-3′,

Three additional probes (TB-groEL1-01, 02 and 03) were also identified. Referring to FIG. 1, TB-groEL1-01 is an oligonucleotide sequence starting at nucleotide # 23,015 and ending at nucleotide # 23,042; TB-groEL1-03 is an oligonucleotide sequence starting at nucleotide # 23,202 and ending at nucleotide # 23,222; and TB-groEL1-06 is an oligonucleotide sequence starting at nucleotide # 23,210 and ending at nucleotide # 23,231. They are as follows:

(SEQ ID NO:6; a.k.a. Tb-groEL1-01)

5′-GACGCGTTCAACACCGCCGACCTAGTCA-3′,

(SEQ ID NO:7; a.k.a. Tb-groEL1-03)

5′-CTTCGGAGAACACGTCGACAC-3′,
and

(SEQ ID NO:8; a.k.a. TB-groEL1-06)

5′-AACACGTCGACACCGAGGACCT-3′.

### Example 2

### Demonstration of Specificity of Probes

The following example is to demonstrate specificity of the probes AGT1051, AGT1052 and AGT1053. The experiments were carried out by labeling genomic nucleic acid samples with a photo-chemically activatable compound. The labeled nucleic acid samples were then hybridized with the probes which were chemically immobilized to magnetic particles. The hybridized materials were detected by chemiluminescence of the label.

Clinical isolates were cultured from patients who were diagnosed with TB. Nucleic acids were isolated from the clinical isolates by lysing cells with Triton X-100 in a Tris-EDTA buffer. Further purification of the nucleic acids was carried out by phenol-chloroform extraction and ethanol precipitation. The precipitated DNA was dissolved in water (1 mg/ml). Similarly, DNA from M. chelonae was also prepared.

The labeling compound was synthesized by the methods described in Dattagupta et al., U.S. Pat. No. 6,242,188 B1. The compound APA was synthesized by following the procedure described in Example 17 of U.S. Pat. No. 6,242,188 except in step 5 of the synthesis, methyl flurosulfonate succinimdo acridine (described in Example 19, step 3) was used instead of biotin compound. The compound was dissolved in ethanol (10 mg/ml).

Ten μl of APA was added to 100 μl of DNA solution (1 mg/ml). The solution was irradiated for 20 minutes using a hand held long wavelength (365 nm) UV lamp at room temperature. After the labeling reaction, excess unbound APA was removed by ethanol precipitation. The labeled sample was then hybridized with immobilized probes at 83° C. for 10 minutes in hybridization buffer (100 mM NaCl, 3% triton X-102, 50 mM PIPES, pH 6.5) and washed 4 times using the wash buffer (20 mM NaCl, 3% triton X-102, 50 mM PIPES, pH 6.5) at the hybridization temperature. The hybrid was detected by chemiluminescence from acridinium ester label in a commercial luminometer (Zylux, Maryville, Tenn.).

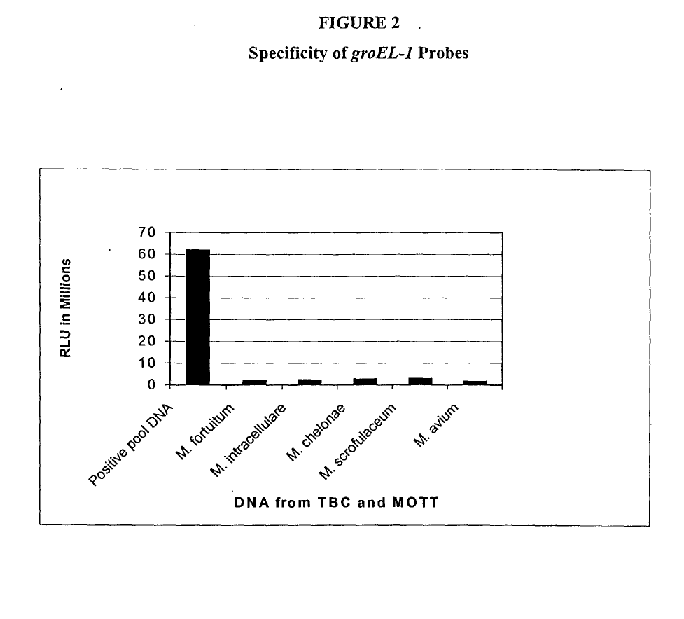

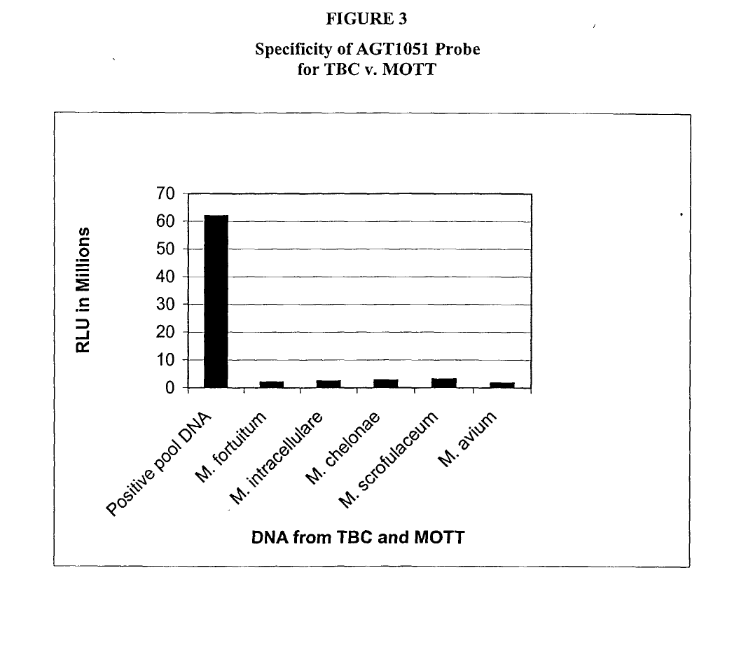

The ability of the oligonucleotide probes to detect the presence of TBC was tested using APA-labeled DNA from Mtb as a positive control and that from M. chelonae as a negative control. The results for AGT1051, AGT1052 and AGT1053 are shown in FIG. 2, and demonstrate that all three probes were specific for TBC. The same experiment was performed to demonstrate the specificity of AGT1051 for positive Mtb pooled DNA compared to five different MOTT species. These results are present in FIG. 3 and demonstrate that AGT1051 clearly distinguishes Mtb from MOTT.

### Example 3

### PCR and Hybridization of Probes

Virtual PCR is conducted with any known software, e.g., Primer III (www.genome.wi.mit.edu). One set of primers for PCR is designed from the groEL-1 gene sequence. Sequences of the left-end and the right-end primers are 5′-CTTCTGCCTTGGCCGGCTTG-3′ (SEQ ID NO:15) and 5′-GCAAGGCGCTGACCGAACTG-3′ (SEQ ID NO:16), respectively. These primers are checked for self-dimerization and hairpin formation abilities using an online program such as Oligo Analyzer ver 2.5 (www.idtdna.com). An amplification reaction is set up with 10 μL of M. tuberculosis H37Rv (ATCC 27294) target DNA (10 pg/μL), 1.5 μL of primer (SEQ ID NO: 15, 10 pmol/μL), 1.5 uL of primer (SEQ ID NO: 16, 10 pmol/μL), 2 μL of 2.5 mM dNTP, 2.5 μL of 10× reaction buffer, and 0.2 uL of Taq DNA polymerase (5 U/μL) in a 25 μL of final mixture. PCR is carried out in a thermal cycler with the following conditions: 94° C. for 5 minutes followed by 28 cycles of [94° C. for 1 min, 65° C. for 1 min, 72° C. for 1 min] followed by 72° C. for 10 min. The reaction mixture reveals the presence of a ˜300 bp amplicon (PCR product) by agarose gel electrophoresis. The PCR product is purified using QIAquick PCR purification kit (QIAGEN, CA, Cat. #28104). Probes AGT01051, AGT01052 and AGT01053 are commercially biotinylated (GenBase, CA). Under the hybridization conditions described in the Example 2, each of the three biotinylated oligonucleotide probes hybridizes with the immobilized purified PCR product.

Similarly, another set of primers for PCR is designed from the groEL-1 gene sequence. Sequences of these primers are as follows: left-end: 5′-TTGACCACCACCGAGCCGTC-3′ (SEQ ID NO:17) and right-end: 5′-TGGACAAGCTGGCCGACACC-3′ (SEQ ID NO:18). These primers are checked for self-dimerization and hairpin formation abilities using online program such as Oligo Analyzer ver 2.5. An amplification reaction is set up with 10 μL of M. tuberculosis H37Rv (ATCC 27294) target DNA (10 pg/μL), 1.5 μL of primer (SEQ ID NO: 17, 10 pmol/μL), 1.5 μL of primer (SEQ ID NO: 18, 10 pmol/μL), 2 uL of 2.5 mM dNTP, 2.5 uL of 10× reaction buffer, and 0.2 μL of Taq DNA polymerase (5U/μL) in a 25 μL of final mixture. PCR is carried out in a thermal cycler with the following conditions: 94° C. for 5 min followed by 28 cycles of [94° C. for 1 min, 65° C. for 1 min, 72° C. for 1 min] followed by 72° C. for 10 min. The reaction mixture reveals the presence of ˜1.3 kb amplicon by agarose gel electrophoresis. The PCR product is purified using QIAquick PCR purification kit (QIAGEN, CA, Cat. #28104). Under the hybridization conditions as described in Example 2, the biotinylated probe AGT01052 hybridizes with the immobilized purified PCR product.

The examples set forth above are provided to give those of ordinary skill in the art with a complete disclosure and description of how to make and use the preferred embodiments of the compositions, and are not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. An oligonucleotide probe for detecting Mycobacterium tuberculosis complex (TBC) in a sample comprising a nucleotide sequence having at least 90% identity with a sequnce selected from the group consisting of:
(SEQ ID NO:3)
5′-TCAACACCGCCGACCTAGTCA-3′,
(SEQ ID NO:4)
5′-CTTCGGAGAACACGTCGACA-3′,
(SEQ ID NO:5)
5′-TCAGGGTGTTCACGTTCAGCCCAT-3′,
(SEQ ID NO:6)
5′-GACGCGTTCAACACCGCCGACCTAGTCA-3′,
(SEQ ID NO:7)
5′-CTTCGGAGAACACGTCGACAC-3′,
(SEQ ID NO:8)
5′-AACACGTCGACACCGAGGACCT-3′,
(SEQ ID NO:9)
3′-AGTTGTGGCGGCTGGATCAGT-5′,
(SEQ ID NO:10)
3′-GAAGCCTCTTGTGCAGCTGT-5′,
(SEQ ID NO:11)
3′-AGTCCCACAAGTGCAAGTCGGGTA-5′,
(SEQ ID NO:12)
3′-CTGCGCAAGTTGTGGCGGCTGGATCAGT-5′,
(SEQ ID NO:13)
3′-GAAGCCTCTTGTGCAGCTGTG-5′,
and
(SEQ ID NO:14)
3′-TTGTGCAGCTGTGGCTCCTGGA-5′.

2. The oligonucleotide probe of claim 1, further comprising a nucleotide sequence having 100% identity with a sequence selected from the group consisting of:
(SEQ ID NO:3)
5′-TCAACACCGCCGACCTAGTCA-3′,
(SEQ ID NO:4)
5′-CTTCGGAGAACACGTCGACA-3′,
(SEQ ID NO:5)
5′-TCAGGGTGTTCACGTTCAGCCCAT-3′,
(SEQ ID NO:6)
5′-GACGCGTTCAACACCGCCGACCTAGTCA-3′,
(SEQ ID NO:7)
5′-CTTCGGAGAACACGTCGACAC-3′,
(SEQ ID NO:8)
5′-AACACGTCGACACCGAGGACCT-3′,
(SEQ ID NO:9)
3′-AGTTGTGGCGGCTGGATCAGT-5′,
(SEQ ID NO:10)
3′-GAAGCCTCTTGTGCAGCTGT-5′,
(SEQ ID NO:11)
3′-AGTCCCACAAGTGCAAGTCGGGTA-5′,
(SEQ ID NO:12)
3′-CTGCGCAAGTTGTGGCGGCTGGATCAGT-5′,
(SEQ ID NO:13)
3′-GAAGCCTCTTGTGCAGCTGTG-5′,
and
(SEQ ID NO:14)
3′-TTGTGCAGCTGTGGCTCCTGGA-5′.

3. The probe of claim 1, which comprises DNA, RNA, PNA or a derivative thereof.

4. The probe of claim 1, which comprises both DNA and RNA or derivatives thereof.

5. The probe of claim 1, which is labeled.

6. The probe of claim 5, wherein the label is selected from the group consisting of a chemical, an enzymatic, an immunogenic, a radioactive, a fluorescent, a luminescent and a FRET label.

7. An array of oligonucleotide probes immobilized on a support for detecting TBC, which array comprises a support suitable for use in nucleic acid hybridization having immobilized thereon a plurality of oligonucleotide probes, at least one of said probes being a probe according to claim 1.

8. The array of claim 7, wherein the plurality of probes comprise DNA, RNA, PNA or a derivative thereof.

9. The array of claim 7, wherein at least one of the probes comprises both DNA and RNA or derivatives thereof.

10. An array of oligonucleotide probes immobilized on a support for detecting TBC, which array comprises a support suitable for use in nucleic acid hybridization having immobilized thereon a plurality of oligonucleotide probes, at least one of said probes being a probe according to claim 2.

11. The array of claim 7, wherein at least one of the probes is labeled.

12. The array of claim 11, wherein the label is selected from the group consisting of a chemical, an enzymatic, an immunogenic, a radioactive, a fluorescent, a luminescent and a FRET label.

13. The array of claim 7, wherein the support comprises a surface that is selected from the group consisting of a silicon, a plastic, a glass, a ceramic, a rubber, and a polymer surface.

14. A method for detecting TBC in a sample, comprising the steps of:
a) providing an oligonucleotide probe comprising a nucleotide sequence, or a complementary strand thereof, having at least 90% identity with a sequence selected from the group consisting of:

| | |
|---|---|
| 5′-TCAACACCGCCGACCTAGTCA-3′, | (SEQ ID NO:3) |
| 5′-CTTCGGAGAACACGTCGACA-3′, | (SEQ ID NO:4) |
| 5′-TCAGGGTGTTCACGTTCAGCCCAT-3′, | (SEQ ID NO:5) |
| 5′-GACGCGTTCAACACCGCCGACCTAGTCA-3′, | (SEQ ID NO:6) |
| 5′-CTTCGGAGAACACGTCGACAC-3′, | (SEQ ID NO:7) |
| 5′-AACACGTCGACACCGAGGACCT-3′, | (SEQ ID NO:8) |
| 3′-AGTTGTGGCGGCTGGATCAGT-5′, | (SEQ ID NO:9) |
| 3′-GAAGCCTCTTGTGCAGCTGT-5′, | (SEQ ID NO:10) |
| 3′-AGTCCCACAAGTGCAAGTCGGGTA-5′, | (SEQ ID NO:11) |
| 3′-CTGCGCAAGTTGTGGCGGCTGGATCAGT-5′, | (SEQ ID NO:12) |
| 3′-GAAGCCTCTTGTGCAGCTGTG-5′, | (SEQ ID NO:13) |
| and | |
| 3′-TTGTGCAGCTGTGGCTCCTGGA-5′. | (SEQ ID NO:14) |

b) contacting said probe with a sample containing or suspected of containing a TBC target nucleotide sequence under conditions suitable for hybridization between said probe and said target nucleotide sequence; and
c) assessing hybridization between said probe and said target nucleotide sequence to detect said TBC in said sample.

15. The method of claim 14, wherein the probe comprises DNA, RNA, PNA or a derivative thereof.

16. The method of claim 14, wherein the probe comprises both DNA and RNA or derivatives thereof.

17. The method of claim 14, wherein the probe comprises a nucleotide sequence that is selected from the group consisting of:

| | |
|---|---|
| 5′-TCAACACCGCCGACCTAGTCA-3′, | (SEQ ID NO:3) |
| 5′-CTTCGGAGAACACGTCGACA-3′, | (SEQ ID NO:4) |
| 5′-TCAGGGTGTTCACGTTCAGCCCAT-3′, | (SEQ ID NO:5) |
| 5′-GACGCGTTCAACACCGCCGACCTAGTCA-3′, | (SEQ ID NO:6) |
| 5′-CTTCGGAGAACACGTCGACAC-3′, | (SEQ ID NO:7) |
| 5′-AACACGTCGACACCGAGGACCT-3′, | (SEQ ID NO:8) |
| 3′-AGTTGTGGCGGCTGGATCAGT-5′, | (SEQ ID NO:9) |
| 3′-GAAGCCTCTTGTGCAGCTGT-5′, | (SEQ ID NO:10) |
| 3′-AGTCCCACAAGTGCAAGTCGGGTA-5′, | (SEQ ID NO:11) |
| 3′-CTGCGCAAGTTGTGGCGGCTGGATCAGT-5′, | (SEQ ID NO:12) |
| 3′-GAAGCCTCTTGTGCAGCTGTG-5′, | (SEQ ID NO:13) |
| and | |
| 3′-TTGTGCAGCTGTGGCTCCTGGA-5′. | (SEQ ID NO:14) |

18. The method of claim 14, wherein the probe or the TBC target nucleotide sequence is labeled.

19. The method of claim 18, wherein the label is selected from the group consisting of a chemical, an enzymatic, an immunogenic, a radioactive, a fluorescent, a luminescent and a FRET label.

20. The method of claim 14, wherein the probe or the TBC target nucleotide sequence is immobilized on a support.

21. The method of claim 14, wherein a plurality of the probes immobilized on a support is used.

22. The method of claim 14, wherein a plurality of samples is assayed.

23. The method of claim 22, wherein the plurality of samples is assayed simultaneously.

24. The method of claim 14, wherein a sample of human origin is assayed.

25. The method of claim 14, wherein the sample is selected from the group consisting of sputum, urine, blood, tissue section, food, soil and water sample.

26. A method for detecting TBC in a sample comprising the steps of:
a) providing an oligonucleotide probe comprising a nucleotide sequence that hybridizes with a target nucleotide sequence, wherein the target nucleotide sequence is all or part of the groEL-1 gene (SEQ ID NO:1), or a complementary strand thereof, and wherein the probe has a G+C content from about 30 to 70%, a Tm value from about 55 to 90° C., a length of at least 8 nucleotides and does not contain any hairpin secondary structure;
b) contacting said probe with a sample containing or suspected of containing a TBC target nucleotide sequence under conditions suitable for hybridization between said probe and said target nucleotide sequence; and
c) assessing hybridization between said probe and said target nucleotide sequence to detect said TBC in said sample.

27. A method for detecting pathogenic bacteria that excrete at least one chaperonin protein of the groE family, said method comprising the steps of:
a) providing an oligonucleotide probe comprising a nucleotide sequence that hybridizes under high stringency with a nucleotide sequence encoding a groE chaperonin protein, wherein the probe has a G+C content from about 30 to 70%, a Tm value from about 55 to 90° C., a length of at least 8 nucleotides and does not contain any hairpin secondary structure;
b) contacting said probe with a sample containing or suspected of containing said pathogenic bacteria under conditions suitable for hybridization between said probe and said nucleotide sequence encoding a groE chaperonin protein; and
c) assessing hybridization between said probe and said nucleotide sequence to detect said pathogenic bacteria in said sample.

28. A method for detecting TBC in a sample comprising the steps of:
a) providing an oligonucleotide probe comprising a nucleotide sequence that hybridizes under high stringency with a target comprising a nucleotide sequence selected from the group consisting of:

| | |
|---|---|
| 5′-TCAACACCGCCGACCTAGTCA-3′, | (SEQ ID NO:3) |
| 5′-CTTCGGAGAACACGTCGACA-3′, | (SEQ ID NO:4) |
| 5′-TCAGGGTGTTCACGTTCAGCCCAT-3′, | (SEQ ID NO:5) |
| 5′-GACGCGTTCAACACCGCCGACCTAGTCA-3′, | (SEQ ID NO:6) |
| 5′-CTTCGGAGAACACGTCGACAC-3′, | (SEQ ID NO:7) |
| 5′-AACACGTCGACACCGAGGACCT-3′, | (SEQ ID NO:8) |
| 3′-AGTTGTGGCGGCTGGATCAGT-5′, | (SEQ ID NO:9) |
| 3′-GAAGCCTCTTGTGCAGCTGT-5′, | (SEQ ID NO:10) |
| 3′-AGTCCCACAAGTGCAAGTCGGGTA-5′, | (SEQ ID NO:11) |
| 3′-CTGCGCAAGTTGTGGCGGCTGGATCAGT-5′, | (SEQ ID NO:12) |
| 3′-GAAGCCTCTTGTGCAGCTGTG-5′, | (SEQ ID NO:13) |
| and | |
| 3′-TTGTGCAGCTGTGGCTCCTGGA-5′; | (SEQ ID NO:14) |

b) contacting said probe with a sample containing or suspected of containing a TBC target nucleotide sequence under conditions suitable for hybridization between said probe and said target nucleotide sequence; and
c) assessing hybridization between said probe and said target nucleotide sequence to detect said TBC in said sample.

29. An oligonucleotide probe for detecting TBC in a sample comprising a nucleotide sequence that hybridizes with a target nucleotide sequence, or a complementary strand thereof as follows:
(SEQ ID NO:2)
5-TCAGTGCGCGTGCCCGTGGTGATGGTCGTGATCTTCTGCCTTGGCCGGCTTGTCGACC
ACGACCGTCTCGGTGGTGAGTACCATCCGGGCAACCGATGACGCGTTCAACACCGCCGAC
CTAGTCACCTTGACCGGGTCGATGACGCCGTCAGCGGCCAAGTCACCATAGCTCAGGGTG
TTCACGTTCAGCCCATGCCCGGCGGGTAGCTCGCTGACCTTGTTGACCACCACCGAGCCG
TCCAAGCCAGCGTTGGCGGCGATCCAGAACAACGGCGCGGCAAGGGCTTCGGAGAACACG
TCGACACCGAGGACCTCGTCACCGGTCAGCGACGC-3′
wherein the probe has a G+C content from about 30 to 70%, a Tm value from about 55 to 90° C., a length of at least 8 nucleotides and does not contain any hairpin secondary structure.

30. The probe of claim 28, which comprises DNA, RNA, PNA or a derivative thereof.

31. The probe of claim 28, which comprises both DNA and RNA or derivatives thereof.

32. The probe of claim 28, which is labeled.

33. The probe of claim 31, wherein the label is selected from the group consisting of a chemical, an enzymatic, an immunogenic, a radioactive, a fluorescent, a luminescent and a FRET label.
